(19) United States
(12) Patent Application Publication
(10) Pub. No.: US 2015/0191638 A1
(43) Pub. Date: Jul. 9, 2015

(54) CATALYSTS AND METHODS FOR ALCOHOL DEHYDRATION

(71) Applicant: Dow Global Technologies LLC, Midland, MI (US)

(72) Inventors: David G. Barton, Midland, MI (US); Adam Chojecki, Gent (BE); Paul R. Elowe, Midland, MI (US); Beata A. Kilos, Midland, MI (US)

(73) Assignee: DOW GLOBAL TECHNOLOGIES LLC, Midland, MI (US)

(21) Appl. No.: 14/417,414

(22) PCT Filed: Aug. 21, 2013
(86) PCT No.: PCT/US13/55961

§ 371 Date: Jan. 26, 2015

Related U.S. Application Data
(60) Provisional application No. 61/694,832, filed on Aug. 30, 2012.

(51) Int. Cl. C09K 5/00; C07C 41/09; B01J 23/10

(52) U.S. Cl. CPC C09K 5/00; B01J 23/10; C07C 41/09

(57) ABSTRACT

Provided is a process for preparing a diaryl ether compound through the dehydration of an aromatic alcohol compound in the presence of a dehydration catalyst. The dehydration catalyst comprises a mixture of two or more of (a) an oxide of a light rare earth element, (b) an oxide of a medium rare earth element, (c) an oxide of a heavy rare earth element, or (d) an oxide of yttrium.

### CROSS-REFERENCE TO RELATED APPLICATIONS

This application claims priority from provisional application Ser. No. 61/694,832, filed Aug. 30, 2012, which is incorporated herein by reference in its entirety.

### BACKGROUND

This invention relates generally to catalysts and methods for the dehydration of aromatic alcohol compounds to ethers. More particularly, the invention uses, for the dehydration of aromatic alcohol compounds to diaryl ethers, a dehydration catalyst comprising a mixture of two or more of (a) an oxide of a light rare earth element, (b) an oxide of a medium rare earth element, (c) an oxide of a heavy rare earth element, or (d) an oxide of yttrium.

Diaryl ethers are an important class of industrial materials. Diphenyl oxide (DPO), for instance, has many uses, most notably as the major component of the eutectic mixture of DPO and biphenyl, which is the standard heat transfer fluid for the concentrating solar power (CSP) industry. With the current boom in CSP has come a tightening of the supply of DPO globally and questions surrounding the sustainability of the technology have arisen.

Diaryl ethers are currently manufactured commercially via two major routes: reaction of a haloaryl compound with an aryl alcohol; or gas-phase dehydration of an aryl alcohol. The first route, for example where chlorobenzene reacts with phenol in the presence of caustic and a copper catalyst, typically leads to less pure product and requires high pressure (5000 psig), uses an expensive alloy reactor and produces stoichiometric quantities of sodium chloride.

The second route, which is a more desirable approach, accounts for the largest volume of diaryl ethers produced but requires a very active and selective catalytic material. For instance, DPO can be manufactured by the gas-phase dehydration of phenol over a thorium oxide (thoria) catalyst (e.g., U.S. Pat. No. 5,925,798). A major drawback of thoria however is its radioactive nature, which makes its handling difficult and potentially costly. Furthermore, the supply of thoria globally has been largely unavailable in recent years putting at risk existing DPO manufacturers utilizing this technology. Additionally, other catalysts for the gas-phase dehydration of phenol, such as zeolite catalysts, titanium oxide, zirconium oxide and tungsten oxide, generally suffer from lower activity, significantly higher impurity content and fast catalyst deactivation.

With a chronic shortage of diaryl ethers such as DPO in sight and a pressing need to increase capacity, it has become crucial to develop alternate methods to produce such materials in a cost-effective and sustainable manner.

The problem addressed by this invention, therefore, is the provision of new catalysts and methods for manufacture of diaryl ethers from aryl alcohol compounds.

### STATEMENT OF THE INVENTION

We have now found that a catalyst comprising a mixture of metal oxides is effective for the preparation of diaryl ethers from aromatic alcohol compounds. Advantageously, the catalyst exhibits remarkable selectivity for the desired product. Moreover, the catalyst is non-radioactive. This invention, therefore, represents a unique solution for diaryl ether supply issues globally.

In one aspect, there is provided a method for preparing a diaryl ether, the method comprising dehydrating an aromatic alcohol compound over a dehydration catalyst, wherein the dehydration catalyst comprises a mixture of two or more of (a) an oxide of a light rare earth element, (b) an oxide of a medium rare earth element, (c) an oxide of a heavy rare earth element, or (d) an oxide of yttrium.

In another aspect, there is provided a method for producing a heat transfer fluid, the method comprising: preparing a diaryl ether by contacting an aromatic alcohol compound with a dehydration catalyst, wherein the dehydration catalyst comprises a mixture of two or more of (a) an oxide of a light rare earth element, (b) an oxide of a medium rare earth element, (c) an oxide of a heavy rare earth element, or (d) an oxide of yttrium; isolating the diaryl ether from the dehydration catalyst; and mixing the isolated diaryl ether with biphenyl such that the mixture forms a eutectic mixture.

### DETAILED DESCRIPTION

Unless otherwise indicated, numeric ranges, for instance as in “from 2 to 10,” are inclusive of the numbers defining the range (e.g., 2 and 10).

Unless otherwise indicated, ratios, percentages, parts, and the like are by weight.

As noted above, in one aspect the invention provides a method for producing a diaryl ether by dehydrating an aromatic alcohol compound over a dehydration catalyst comprising a mixture of two or more of (a) an oxide of a light rare earth element, (b) an oxide of a medium rare earth element, (c) an oxide of a heavy rare earth element, or (d) an oxide of yttrium. It has been discovered that such catalysts exhibit high selectivity for the desired diaryl ether compounds with relatively low formation of undesirable byproducts. For instance, as demonstrated by the examples, in the synthesis of diphenyl oxide from phenol, a selectivity for the DPO of 50% or greater may be achieved. In some embodiments, a selectivity of 80% or greater may be achieved. In some embodiments, a selectivity of 90% or greater, or 95% or greater is possible.

In addition to being highly selective, the catalysts are also advantageous because they are non-radioactive, thus eliminating the safety and environmental issues, as well as higher costs, associated with the handling of radioactive materials, such as the thoria catalysts of the prior art.

The dehydration catalyst of the invention comprises a mixture of two or more of (a) an oxide of a light rare earth element, (b) an oxide of a medium rare earth element, (c) an oxide of a heavy rare earth element, or (d) an oxide of yttrium. By a “light rare earth element” is meant lanthanum, cerium, praseodymium, neodymium, or mixtures of two or more thereof. By “oxide of a light rare earth element” is meant a compound that contains at least one oxygen-light rare earth element chemical bond. Examples include lanthanum oxide (La₂O₃), cerium oxide (CeO₂), praseodymium oxide (e.g., PrO₂, Pr₂O₃, Pr₆O₁₁, or mixtures), and neodymium oxide (Nd₂O₃). By a “medium rare earth element” is meant samarium, europium, gadolinium, or mixtures thereof. By “oxide of medium rare earth element” is meant a compound that contains at least one oxygen-medium rare earth element bond. Examples include Sm₂O₃, EU₂O₃, and Gd₂O₃. By a “heavy rare earth element” is meant terbium, dysprosium, holmium, erbium, thulium, ytterbium, lutetium, or mixtures thereof. By “oxide of heavy rare earth element” is meant a compound that contains at least one oxygen-heavy rare earth element bond. Examples include, but are not limited to, Tb₂O₃, Tb₄O₇, TbO₂, Tb₆O₁₁, Dy₂O₃, Ho₂O₃, Er₂O₃, Tm₂O₃, Yb₂O₃, and Lu₂O₃. By “oxide of yttrium” is meant a compound that contains at least yttrium and oxygen atoms. An example is yttrium oxide (yttria). Each of (a), (b), (c), and (d) may also be referred to herein as a “metal oxide.”

As noted, the dehydration catalyst of the invention comprises a mixture of two or more of (a) an oxide of a light rare earth element, (b) an oxide of a medium rare earth element, (c) an oxide of a heavy rare earth element, or (d) an oxide of yttrium. In some embodiments, the dehydration catalyst comprises a mixture of (a) and (b), alternatively a mixture of (a) and (c), alternatively a mixture of (a) and (d), alternatively a mixture of (b) and (c), alternatively a mixture of (b) and (d), or alternatively a mixture of (c) and (d). In some embodiments, the dehydration catalyst comprises a mixture of (a), (b), and (c), alternatively a mixture of (a), (b), and (d), alternatively a mixture of (a), (c), (d), or alternatively a mixture of (b), (c), and (d). In some embodiments, the dehydration catalyst comprises a mixture of (a), (b), (c), and (d).

One or more of the metal oxides of which the dehydration catalyst is comprised may optionally contain other atoms, such as halogens, for instance chloride or fluoride. In some embodiments, a preferred catalyst for use in the invention contains a metal oxide as described above and chlorine atoms. In some embodiments, the catalyst comprises chlorine (in addition to the metal oxide) in an amount of less than 54 weight percent, alternatively 40 weight percent or less, alternatively 20 weight percent or less, alternatively 10 weight percent or less, or alternatively 2 weight percent or less. In some embodiments, the catalyst comprises the chlorine in an amount of at least 0.001 weight percent, alternatively at least 0.1 weight percent, alternatively at least 1 weight percent, or alternatively at least 2 weight percent. In some embodiments, the catalyst contains between 1 and 20 weight percent chlorine. The chlorine may be in the form of chloride ion (Cl⁻).

Non limiting examples of suitable compounds for the mixture described above may include samarium oxychloride, europium oxychloride, gadolinium oxychloride, yttrium oxychloride, lanthanum oxychloride, praseodymium oxychloride, neodymium oxychloride, terbium oxychloride, dysprosium oxychloride, holmium oxychloride, erbium oxychloride, thulium oxychloride, cerium oxychloride, ytterbium oxychloride, lutetium oxychloride. By “oxychloride” is meant a compound that contains metal-oxygen and metal-chlorine bonds. Non-limiting examples also include physical mixtures of a metal oxide together with a compound containing chlorine, such as NH₄Cl, HCl, or a metal chloride, such as yttrium chloride. Examples further include, again without limitation, metal oxide catalysts based on chlorate oxyanions, such as hypochlorite (ClO⁻); chlorite (ClO₂⁻); chlorate (ClO₃⁻), perchlorate (ClO₄⁻) where Cl is oxidized (+2, +3, +4, +5), as well as amorphous materials.

It should be noted that each of (a), (b), (c), (d) described above may themselves be present as mixtures of oxides, oxychlorides, etc. (e.g., mixture of light rare earth oxides for (a)). By way of illustration, (a) may be a mixture of oxides or oxychlorides of lanthanum, cerium, praseodymium, and neodymium. By way of further illustration, (b) may be a mixture of oxides or oxychlorides of samarium, europium and gadolinium. By way of still further illustration, (c) may be a mixture of oxides or oxychlorides of terbium, dysprosium, holmium, erbium, thulium, ytterbium, and lutetium.

In some embodiments, (a) may be a mixture of oxides or oxychlorides of lanthanum, praseodymium, and neodymium. In some embodiments, (b) may be a mixture of oxides or oxychlorides of samarium and gadolinium. In some embodiments, (c) may be a mixture of oxides or oxychlorides of terbium, dysprosium, holmium, erbium, ytterbium, and lutetium.

In some embodiments, the dehydration catalyst comprises a mixture of oxides or oxychlorides, preferably oxychlorides, of lanthanum, praseodymium, neodymium, samarium, gadolinium, terbium, dysprosium, holmium, erbium, ytterbium, lutetium, cerium, thullium, europium, and yttrium.

In some embodiments, the dehydration catalyst comprises a mixture of oxides or oxychlorides, preferably oxychlorides, of lanthanum, praseodymium, neodymium, samarium, gadolinium, terbium, dysprosium, holmium, erbium, ytterbium, lutetium, and yttrium.

In some embodiments, the dehydration catalyst comprises a mixture of oxides or oxychlorides, preferably oxychlorides, of lanthanum and yttrium.

In some embodiments, the dehydration catalyst comprises a mixture of oxides or oxychlorides, preferably oxychlorides, of lanthanum and gadolinium.

In some embodiments, the dehydration catalyst comprises a mixture of oxides or oxychlorides, preferably oxychlorides, of lanthanum and ytterbium.

There is no particular limitation in the invention on the relative amounts of the individual active components of the catalyst relative to each other. In some embodiments, it may be preferred to use an equimolar amount of the active components. It should further be noted that in using a mixture of active components, one of the advantages of the invention is that it is not necessary to isolate and/or purify an individual catalytic compound, or to enrich a particular catalytic compound, thus potentially reducing costs.

Catalysts suitable for use in the invention may be prepared by those skilled in the art or they may be purchased from commercial vendors.

The catalyst may optionally contain a binder and/or matrix material that is different from the active material. Non-limiting examples of binders that are useful alone or in combination include various types of hydrated alumina, silicas and/or other inorganic oxide sols, and carbon. Upon heating, the inorganic oxide sol, preferably having a low viscosity, is converted into an inorganic oxide binder component.

Where the catalyst composition contains a matrix material, this is preferably different from the active catalyst and any binder. Non-limiting examples of matrix materials include clays or clay-type compositions.

The catalyst, including any binder or matrix materials, may be unsupported or supported. Non-limiting examples of suitable support materials include titania, alumina, zirconia, silica, carbons, zeolites, magnesium oxide, and mixtures thereof. In some embodiments, the support material may itself be an active metal oxide. An example is lanthanum oxide. In some embodiments, the dehydration catalyst comprises an oxide or oxychloride of yttrium impregnated on a lanthanum oxide support. In some embodiments, the dehydration catalyst comprises an oxide or oxychloride of ytterbium impregnated on a lanthanum oxide support. In some embodiments, the dehydration catalyst comprises an oxide or oxychloride of gadolinium impregnated on a lanthanum oxide support.

Where the catalyst contains a binder, matrix or support material, the amount of the active components of the catalyst may be between 1 and 99 percent by weight based on the total weight of the catalyst (including the active component, and any support, binder or matrix materials).

The catalyst may be formed into various shapes and sizes for ease of handling. For instance, the catalyst (plus any binder, matrix, or support) may be in the form of pellets, spheres, or other shapes commonly used in the industry.

Aromatic alcohol compounds suitable for use in the process of this invention include aromatic compounds containing at least one alcohol group and one, two, three or more aromatic moieties. Suitable compounds include phenols and α- and β-hydroxy-substituted fused aromatic ring systems. Apart from the hydroxy substituent, the compounds may be unsubstituted, as in phenol or naphthol. Optionally, however, the compounds may be further substituted with at least one alkyl group containing from 1 to about 10 carbon atoms, preferably, from 1 to 3 carbon atoms, or substituted with at least one alternative substituent which is inert to the dehydration coupling reaction. Suitable inert substituents include cyano, amino, nitro, carboxylic acid (e.g., C₀-C₆—COOH), ester, C₆-C₁₂ aryl, C₂-C₆ alkenyl, alkyloxy, aryloxy, and phenoxy moieties. It is also possible for the aromatic alcohol compound to be substituted with both an alkyl substituent and one of the alternative inert substituents. Each of the aforementioned alkyl substituents and/or alternative inert substituents is attached preferably to an aromatic ring carbon atom which is located in an ortho, meta or para position relative to the hydroxy moiety. Optionally, the alkyl substituent may contain from 3 to 4 carbon atoms, and in combination with a phenol or fused aromatic ring system may form a saturated ring fused to the aromatic ring. An acceptable feed may contain a mixture of aromatic alcohols, including mixtures of the foregoing.

Non-limiting examples of suitable phenols include unsubstituted phenol, m-cresol, p-cresol, 3,4-xylenol, 3,5-xylenol, and 3,4,5-trimethylphenol. Other suitable phenols include compounds corresponding to the above-mentioned examples except that one or more of the methyl substituents are replaced by an ethyl, propyl or butyl substituent. Non-limiting examples of α- and β-hydroxy-substituted fused aromatic ring systems include α- and β-naphthol and 5- tetralinol. Other non-limiting examples of aromatic alcohols include benzenediols (catechol, resorcinol or hydroquinone), o-cresol, ophenylphenol, m-phenylphenol or p-phenylphenol. One skilled in the art may find other phenols and α- and β-hydroxy-substituted fused aromatic ring systems which are also suitable for the purposes of this invention. Preferably, the aromatic alcohol is unsubstituted phenol or a substituted phenol wherein the substituent is methyl, ethyl or hydroxyl. More preferably, the aromatic alcohol is unsubstituted phenol, cresol or a benzenediol. Most preferably, the aromatic alcohol is unsubstituted phenol.

According to the method of the invention for preparing a diaryl ether, a dehydration catalyst as described herein is contacted with the aromatic alcohol compound. The contacting of the catalyst with the aromatic alcohol compound is carried out under reaction conditions such that the diaryl ether is formed.

The catalyst is contacted with the aromatic alcohol compound either in the gas phase or in the liquid phase. In addition, the aromatic alcohol may be diluted with a diluent or it may be neat. Suitable diluents include, without limitation, nitrogen, argon, water vapor, water, oxygen or hydrogen. When a diluent is used, the concentration of the aromatic alcohol compound may be, for instance, 1 volume percent or greater and less than 100 volume percent.

In a preferred embodiment, the aromatic alcohol is contacted with the catalyst in the gas phase. Typically, the aromatic alcohol is introduced into a reactor containing the catalyst at elevated temperature, for instance, between 200 and 800° C., alternatively between 300 and 600° C., alternatively between 400 and 600° C., or alternatively between 450 and 550° C. The reaction may be conducted at atmospheric pressure, under reduced pressure, or at elevated pressure such as up to 5000 psi. In some embodiments, atmospheric pressure or slightly above (e.g., up to about 50 psi) is preferred. In some embodiments, the gas flow rate of the aromatic alcohol over the catalyst (weighted hourly space velocity or WHSV) is from 0.01 to 100 grams per gram per hour (g/g-h). In some embodiments, WHSV is from 0.1 to 20 g/g-h, alternatively 0.1 to 5 g/g-h, or alternatively 0.1 to 1 g/g-h.

In some embodiments, it may be useful to subject the reactor to startup conditions which may provide various benefits, such as prolonging catalyst life. Suitable startup condition include, for example, exposing the catalyst to dilute amounts of the aromatic alcohol at lower temperature before changing to full operating conditions as described above and demonstrated by the examples.

Following the reaction, the diaryl ether product is recovered from the catalyst and optionally further purified. Unreacted alcohol and other reaction by-products may be separated using methods known in the art. Such methods include but are not limited to distillation, crystal refining, simulated moving bed technique or a combination thereof.

In some embodiments, the diaryl ether prepared by the process of the invention is diphenyl oxide (DPO). Other diaryl ether compounds that may be prepared by the process of the invention include, without limitation, compounds containing at least one ether functionality whereby two aryl moieties are connected by an oxygen atom (Ar—O—Ar′), including polyaryl compounds and compounds prepared from the aromatic alcohols described above. Specific examples include, but are not limited to, dibenzofuran, phenoxytoluene isomers, including 3-phenoxytoluene, ditolyl ether isomers, polyphenyl ethers (PPEs), biphenylphenyl ether isomers and naphthyl phenyl ethers.

The diaryl ethers prepared by the invention are useful in a variety of applications, including as high temperature solvents, as intermediates in preparing flame retardants and surfactants, and as components in heat transfer fluids. Furthermore, certain diaryl ethers prepared by the invention are useful as high performance lubricants and as intermediates in preparing pyrethroid insecticides.

In some embodiments, a preferred use of the diaryl ether is in high temperature heat transfer fluids. High temperature heat transfer fluids may be prepared by making the diaryl ether according to the process described above and then mixing the diaryl ether with biphenyl. The amounts necessary to provide a suitable fluid can be readily determined by a person with ordinary skill in the art. For diphenyl oxide and biphenyl, the amount of DPO may be, for instance, from 70 to 75 weight percent based on the total weight of the DPO and biphenyl. A preferred amount of DPO is that required to form a eutectic mixture with the biphenyl, which is about 73.5 weight percent based on the total weight of the DPO and biphenyl.

Some embodiments of the invention will now be described in detail in the following Examples.

### EXAMPLES

### Example 1

An aqueous mixed metal solution is prepared by dissolving 0.4092 g LaCl₃, 0.1833 g PrCl₃, 0.8678 g NdCl₃, 0.5704 g SmCl₃, 1.2506 g GdCl₃, 0.1790 g TbCl₃, 1.6950 g DyCl₃, 0.3518 g HoCl₃, 1.0316 g ErCl₃, 0.7821 g YbCl₃, 0.1203 g LuCl₃, 23.2823 g YCl₃, and 0.6837 g Al(NO₃)₃ in 98.6 mL DI H₂O, is added dropwise along with ammonium hydroxide (150.4 g, from 29% NH₃ solution) over 15 min into a 600-ml beaker containing an initial 100 ml DI H₂O. The solution is stirred at 500 rpm on a magnetic stir plate with a 2-inch stir bar. The resulting precipitate is allowed to age in solution for 1 h with stirring, after which it is centrifuged at 5000 rpm for 10 min. The decanted precipitate is placed into an oven, dried at 120° C. for 4 h and calcined at 500° C. for 4 h with a ramp rate of 5° C./min to yield the solid product.

### Example 2

The catalyst from Example 1 is used for the dehydration of phenol. The powder is pressed and sieved to obtain particles that are between 0.60 mm and 0.85 mm in diameter. The particles are loaded into an electrically heated stainless steel reactor tube and heated to the reaction temperature with nitrogen flowing through the tube. After the reaction temperature is reached, vapor-phase phenol is passed through the reactor tube. The conversion of phenol is carried out at a weighted hourly space velocity of 1 (WHSV=gram phenol/gram catalyst-hour) and at 500° C. Test conditions and results are shown in Table 1.

TABLE 1

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 12.15% | 97.89% | 0.32% | 1.75% | 0.00% | 0.00% | 0.05% |
| Feed: PhOH | | | | | | | |
| ToS = 1.25 h | | | | | | | |
| WHSV 1 h⁻¹ | | | | | | | |
| T = 500° C. | 14.14% | 96.65% | 0.25% | 3.05% | 0.00% | 0.00% | 0.04% |
| Feed: PhOH | | | | | | | |
| ToS = 2.5 h | | | | | | | |
| WHSV 1 h⁻¹ | | | | | | | |
| T = 500° C. | 13.55% | 96.46% | 0.81% | 2.61% | 0.00% | 0.05% | 0.07% |
| Feed: PhOH | | | | | | | |
| ToS = 4.75 h | | | | | | | |
| WHSV 1 h⁻¹ | | | | | | | |
| T = 500° C. | 13.72% | 96.12% | 0.77% | 2.85% | 0.02% | 0.07% | 0.17% |
| Feed: PhOH | | | | | | | |
| ToS = 6.5 h | | | | | | | |
| WHSV 1 h⁻¹ | | | | | | | |

OPP: orthophenylphenol.
DBF: dibenzofuran.
O-BIPPE: ortho-biphenylphenyl ether.
M-BIPPE: meta-biphenylphenyl ether.
P-BIPPE: para-biphenylphenyl ether.
PhOH: phenol.
N2: nitrogen.
ToS: time on stream (ToS = 0 hours defined as start of phenol flow).

### Example 3

An aqueous lanthanum and yttrium mixed metal solution, prepared by dissolving 9.2814 g LaCl₃ and 7.5833 g YCl₃, in 50 ml DI H₂O, is added dropwise along with ammonium hydroxide (18.1 g, from 29% NH₃ solution) over 15 min into a 600-ml beaker containing an initial 100 ml DI H₂O. The solution is stirred at 500 rpm on a magnetic stir plate with a 2-inch stir bar. The resulting precipitate is allowed to age in solution for 1 h with stirring, after which it is centrifuged at 5000 rpm for 10 min. The decanted precipitate is placed into an oven, dried at 120° C. for 4 h and calcined at 500° C. for 4 h with a ramp rate of 5° C./min to yield the solid product.

### Example 4

The catalyst from Example 3 is used for the dehydration of phenol. The powder is pressed and sieved to obtain particles that are between 0.60 mm and 0.85 mm in diameter. The particles are loaded into an electrically heated stainless steel reactor tube and heated to the reaction temperature with nitrogen flowing through the tube. After the reaction temperature is reached, vapor-phase phenol is passed through the reactor tube. The conversion of phenol is carried out at a weighted hourly space velocity of 1 (WHSV=gram phenol/gram catalyst-hour) and at 500° C. Test conditions and results are shown in Table 2.

TABLE 2

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 5.51% | 86.82% | 3.27% | 9.91% | 0.00% | 0.00% | 0.00% |
| Feed: PhOH | | | | | | | |
| ToS = 2.25 h | | | | | | | |
| WHSV 1 h⁻¹ | | | | | | | |
| T = 500° C. | 5.38% | 84.78% | 3.27% | 11.95% | 0.00% | 0.00% | 0.00% |
| Feed: PhOH | | | | | | | |
| ToS = 3.5 h | | | | | | | |
| WHSV 1 h⁻¹ | | | | | | | |
| T = 500° C. | 5.01% | 85.39% | 2.82% | 11.79% | 0.00% | 0.00% | 0.00% |
| Feed: PhOH | | | | | | | |
| ToS = 4.5 h | | | | | | | |
| WHSV 1 h⁻¹ | | | | | | | |

### Example 5

An aqueous lanthanum and gadolinium mixed metal solution, prepared by dissolving 9.2830 g LaCl₃ and 9.2948 g GdCl₃, in 50 ml DI H₂O, is added dropwise along with ammonium hydroxide (18.1 g, from 29% NH₃ solution) over 15 min into a 600-ml beaker containing an initial 100 ml DI H₂O. The solution is stirred at 500 rpm on a magnetic stir plate with a 2-inch stir bar. The resulting precipitate is allowed to age in solution for 1 h with stirring, after which it is centrifuged at 5000 rpm for 10 min. The decanted precipitate is placed into an oven, dried at 120° C. for 4 h and calcined at 500° C. for 4 h with a ramp rate of 5° C./min to yield the solid product.

### Example 6

The catalyst from Example 5 is used for the dehydration of phenol. The powder is pressed and sieved to obtain particles that are between 0.60 mm and 0.85 mm in diameter. The particles are loaded into an electrically heated stainless steel reactor tube and heated to the reaction temperature with nitrogen flowing through the tube. After the reaction temperature is reached, vapor-phase phenol is passed through the reactor tube. The conversion of phenol is carried out at a weighted hourly space velocity of 1 (WHSV=gram phenol/gram catalyst-hour) and at 500° C. Test conditions and results are shown in Table 3.

TABLE 3

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 1.10% | 84.69% | 1.11% | 14.20% | 0.00% | 0.00% | 0.00% |
| Feed: PhOH | | | | | | | |
| ToS = 2.25 h | | | | | | | |
| WHSV 1 h⁻¹ | | | | | | | |
| T = 500° C. | 1.06% | 84.45% | 1.44% | 13.83% | 0.00% | 0.00% | 0.29% |
| Feed: PhOH | | | | | | | |
| ToS = 3.5 h | | | | | | | |
| WHSV 1 h⁻¹ | | | | | | | |
| T = 500° C. | 1.02% | 84.85% | 2.11% | 12.65% | 0.00% | 0.00% | 0.39% |
| Feed: PhOH | | | | | | | |
| ToS = 4.5 h | | | | | | | |
| WHSV 1 h⁻¹ | | | | | | | |

### Example 7

An aqueous lanthanum and ytterbium mixed metal solution, prepared by dissolving 9.2836 g LaCl₃ and 9.6879 g YbCl₃, in 50 ml DI H₂O, is added dropwise along with ammonium hydroxide (18.1 g, from 29% NH₃ solution) over 15 min into a 600-ml beaker containing an initial 100 ml DI H₂O. The solution is stirred at 500 rpm on magnetic stir plate with a 3-inch stir bar. The resulting precipitate is allowed to age in solution for 1 h with stirring, after which it is centrifuged at 5000 rpm for 10 min. The decanted precipitate is placed into an oven, dried at 120° C. for 4 h and calcined at 500° C. for 4 h with a ramp rate of 5° C./min to yield the solid product.

### Example 8

The catalyst from Example 7 is used for the dehydration of phenol. The powder is pressed and sieved to obtain particles that are between 0.60 mm and 0.85 mm in diameter. The particles are loaded into an electrically heated stainless steel reactor tube and heated to the reaction temperature with nitrogen flowing through the tube. After the reaction temperature is reached, vapor-phase phenol is passed through the reactor tube. The conversion of phenol is carried out at a weighted hourly space velocity of 1 (WHSV=gram phenol/gram catalyst-hour) and at 500° C. Test conditions and results are shown in Table 4.

TABLE 4

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 1.49% | 91.26% | 0.11% | 8.17% | 0.00% | 0.00% | 0.46% |
| Feed: PhOH | | | | | | | |
| ToS = 1.5 h | | | | | | | |
| WHSV 1 h⁻¹ | | | | | | | |
| T = 500° C. | 1.37% | 91.48% | 1.46% | 6.42% | 0.00% | 0.00% | 0.63% |
| Feed: PhOH | | | | | | | |
| ToS = 2.5 h | | | | | | | |
| WHSV 1 h⁻¹ | | | | | | | |
| T = 500° C. | 1.52% | 92.28% | 1.62% | 5.58% | 0.00% | 0.00% | 0.53% |
| Feed: PhOH | | | | | | | |
| ToS = 3.75 h | | | | | | | |
| WHSV 1 h⁻¹ | | | | | | | |
| T = 500° C. | 1.68% | 92.00% | 0.49% | 6.98% | 0.00% | 0.00% | 0.53% |
| Feed: PhOH | | | | | | | |
| ToS = 5 h | | | | | | | |
| WHSV 1 h⁻¹ | | | | | | | |

### Example 9

### Preparation of 12 wt % Y on La₂O₃ Mixture

Prior to the impregnation process, a La₂O₃ support prepared by a precipitation method with BET surface area of 20 m/g is calcined at 600° C. for 3 hours in static air. 12 wt % Y on La₂O₃ catalyst is prepared by one-step incipient wetness impregnation of La₂O₃ at ambient temperature. A glass beaker is charged with 5 g of pre-dried La₂O₃. A 10-ml graduated cylinder is loaded with 2.0480 g of YCl₃·6H₂O to yield 12 wt % of Y with 4.5 g of H₂O. The support is impregnated with aqueous solution of yttrium added to the La₂O₃ in small fractions. After each addition, the support is agitated to break up clumps and uniformly disperse yttrium throughout the carrier material. The impregnated sample is then treated at 110° C. for 4 hours in flowing air and then at 500° C. for an additional 4 hours with a 5° C./min ramp.

### Example 10

The catalyst from Example 9 is used for the dehydration of phenol. The powder is pressed and sieved to obtain particles that are between 0.60 mm and 0.85 mm in diameter. The particles are loaded into an electrically heated stainless steel reactor tube and heated to the reaction temperature with nitrogen flowing through the tube. After the reaction temperature is reached, vapor-phase phenol is passed through the reactor tube. The conversion of phenol is carried out at a weighted hourly space velocity of 1 (WHSV=gram phenol/gram catalyst-hour) and at 500° C. Test conditions and results are shown in Table 5.

TABLE 5

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 1.03% | 74.77% | 6.95% | 17.21% | 0.00% | 0.27% | 0.81% |
| Feed: PhOH | | | | | | | |
| ToS = 1.5 h | | | | | | | |
| WHSV 1 h⁻¹ | | | | | | | |
| T = 500° C. | 1.09% | 75.71% | 2.35% | 21.38% | 0.00% | 0.00% | 0.55% |
| Feed: PhOH | | | | | | | |
| ToS = 2.5 h | | | | | | | |
| WHSV 1 h⁻¹ | | | | | | | |
| T = 500° C. | 0.84% | 76.10% | 7.45% | 15.95% | 0.00% | 0.00% | 0.50% |
| Feed: PhOH | | | | | | | |
| ToS = 4 h | | | | | | | |
| WHSV 1 h⁻¹ | | | | | | | |
| T = 500° C. | 0.99% | 75.70% | 6.10% | 17.62% | 0.00% | 0.00% | 0.58% |
| Feed: PhOH | | | | | | | |
| ToS = 5 h | | | | | | | |
| WHSV 1 h⁻¹ | | | | | | | |

### Example 11

### Preparation of 6 wt % Y on La₂O₃ Mixture

Prior to the impregnation process, a La₂O₃ support prepared by a precipitation method with BET surface area of 20 m/g is calcined at 600° C. for 3 hours in static air. 6 wt % Y on La₂O₃ catalyst is prepared by one-step incipient wetness impregnation of the La₂O₃ at ambient temperature. A glass beaker is charged with 3 g of pre-dried La₂O₃. A 5-mL graduated cylinder is loaded with 0.6148 g of YCl₃·6H₂O to yield 6 wt % of Y with 2.821 g of H₂O. The support is impregnated with aqueous solution of yttrium added to the La₂O₃ in small fractions. After each addition, the support is agitated to break up clumps and uniformly disperse yttrium throughout the carrier material. The impregnated sample is then treated at 110° C. for 4 hours in flowing air and then at 500° C. for an additional 4 hours with a 5° C./min ramp.

### Example 12

The catalyst from Example 11 is used for the dehydration of phenol. The powder is pressed and sieved to obtain particles that are between 0.60 mm and 0.85 mm in diameter. The particles are loaded into an electrically heated stainless steel reactor tube and heated to the reaction temperature with nitrogen flowing through the tube. After the reaction temperature is reached, vapor-phase phenol is passed through the reactor tube. The conversion of phenol is carried out at a weighted hourly space velocity of 1 (WHSV=gram phenol/gram catalyst-hour) and at 500° C. Test conditions and results are shown in Table 6.

TABLE 6

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 0.42% | 84.40% | 2.42% | 13.18% | 0.00% | 0.00% | 0.00% |
| Feed: PhOH | | | | | | | |
| ToS = 1.5 h | | | | | | | |
| WHSV 1 h⁻¹ | | | | | | | |
| T = 500° C. | 0.44% | 85.90% | 1.49% | 12.61% | 0.00% | 0.00% | 0.00% |
| Feed: PhOH | | | | | | | |
| ToS = 2.5 h | | | | | | | |
| WHSV 1 h⁻¹ | | | | | | | |
| T = 500° C. | 0.39% | 86.71% | 1.73% | 11.55% | 0.00% | 0.00% | 0.00% |
| Feed: PhOH | | | | | | | |
| ToS = 4 h | | | | | | | |
| WHSV 1 h⁻¹ | | | | | | | |
| T = 500° C. | 0.35% | 87.41% | 1.77% | 10.82% | 0.00% | 0.00% | 0.00% |
| Feed: PhOH | | | | | | | |
| ToS = 5 h | | | | | | | |
| WHSV 1 h⁻¹ | | | | | | | |

### Example 13

### Preparation of 10 wt % Gd on La₂O₃ Mixture

Prior to the impregnation process, a La₂O₃ support prepared by a precipitation method with BET surface area of 20 m²/g is calcined at 600° C. for 3 hours in static air. 10 wt % Gd on La₂O₃ catalyst is prepared by one-step incipient wetness impregnation of the La₂O₃ at ambient temperature. A glass beaker is charged with 5 g of pre-dried La₂O₃. A 10-mL graduated cylinder is loaded with 1.1815 g of GdCl₃·6H₂O to yield 10 wt % of Gd with 4.5 g of H₂O. The support is impregnated with aqueous solution of gadolinium added to the La₂O₃ in small fractions. After each addition, the support is agitated to break up clumps and uniformly disperse gadolinium throughout the carrier material. The impregnated sample is then treated at 20° C. for 4 hours in flowing air and then at 500° C. for an additional 4 hours with a 5° C./min ramp.

### Example 14

The catalyst from Example 13 is used for the dehydration of phenol. The powder is pressed and sieved to obtain particles that are between 0.60 mm and 0.85 mm in diameter. The particles are loaded into an electrically heated stainless steel reactor tube and heated to the reaction temperature with nitrogen flowing through the tube. After the reaction temperature is reached, vapor-phase phenol is passed through the reactor tube. The conversion of phenol is carried out at a weighted hourly space velocity of 1 (WHSV=gram phenol/gram catalyst-hour) and at 500° C. Test conditions and results are shown in Table 7.

TABLE 7

| | Conversion | Selectivity [mol. %] | | | | | |
|---|---|---|---|---|---|---|---|
| Test | [mol. %] | Diphenyl | | | | | |
| Conditions | Phenol | Oxide | OPP | DBF | O-BIPPE | M-BIPPE | P-BIPPE |
| T = 500° C. | 4.40% | 78.93% | 0.65% | 19.56% | 0.00% | 0.49% | 0.36% |
| Feed: PhOH | | | | | | | |
| ToS = 1.75 h | | | | | | | |
| WHSV 1 h⁻¹ | | | | | | | |
| T = 500° C. | 3.24% | 78.71% | 3.76% | 16.81% | 0.00% | 0.34% | 0.39% |
| Feed: PhOH | | | | | | | |
| ToS = 2.5 h | | | | | | | |
| WHSV 1 h⁻¹ | | | | | | | |
| T = 500° C. | 2.77% | 76.77% | 5.89% | 16.55% | 0.00% | 0.39% | 0.40% |
| Feed: PhOH | | | | | | | |
| ToS = 4.75 h | | | | | | | |
| WHSV 1 h⁻¹ | | | | | | | |
| T = 500° C. | 2.83% | 74.36% | 3.02% | 21.78% | 0.00% | 0.38% | 0.45% |
| Feed: PhOH | | | | | | | |
| ToS = 5.5 h | | | | | | | |
| WHSV 1 h⁻¹ | | | | | | | |

1. A method for preparing a diaryl ether, the method comprising dehydrating an aromatic alcohol compound over a dehydration catalyst, wherein the dehydration catalyst comprises a mixture of two or more of (a) an oxide of lanthanum, cerium, praseodymium, neodymium, or mixtures of two or more thereof, (b) an oxide of samarium, europium, gadolinium, or mixtures thereos, (c) an oxide of of terbium, dysprosium, holmium, erbium, thulium, ytterbium, lutetium, or mixtures thereof, or (d) an oxide of yttrium.

2. The method of claim 1 wherein the dehydration catalyst further comprises a halogen.

3. The method of claim 2 wherein the halogen is chloride or fluoride ion.

4. The method of claim 1 wherein the dehydration catalyst further comprises a binder.

5. The method of claim 1 wherein the dehydration catalyst is supported.

6. The method of claim 1 wherein the dehydration catalyst is unsupported.

7. The method of claim 1 wherein the dehydration of the alcohol is conducted at a temperature from 250 to 600° C.

8. The method of claim 1 wherein the alcohol feed is diluted with a diluent.

9. The method of claim 1 wherein the aromatic alcohol compound is phenol and the diaryl ether produced is diphenyl oxide.

10. A method for producing a heat transfer fluid, the method comprising:
preparing a diaryl ether by contacting an aromatic alcohol compound with a dehydration catalyst, wherein the dehydration catalyst comprises a mixture of two or more of (a) an oxide of lanthanum, cerium, praseodymium, neodymium, or mixtures of two or more thereof, (b) an oxide of samarium, europium, gadolinium, or mixtures thereo, (c) an oxide of terbium, dysprosium, holmium, erbium, thulium, ytterbium, lutetium, or mixtures thereof, or (d) an oxide of yttrium;
isolating the diaryl ether from the dehydration catalyst; and
mixing the isolated diaryl ether with biphenyl such that the mixture forms a eutectic mixture.
